(19) United States
(12) Patent Application Publication
(10) Pub. No.: US 2023/0099028 A1
(43) Pub. Date: Mar. 30, 2023

(54) HEART RATE VARIABILITY DETERMINATION

(71) Applicant: Covidien LP, Mansfield, MA (US)

(72) Inventors: Pedro M. Elias Monteiro Gomes, Sobral de Monte Agraco (PT); Scott McGonigle, Loanhead (GB); Dean Montgomery, Edinburgh (GB)

(21) Appl. No.: 17/822,284

(22) Filed: Aug. 25, 2022

Related U.S. Application Data
(60) Provisional application No. 63/250,775, filed on Sep. 30, 2021.

(51) Int. Cl. A61B 5/024; A61B 5/00

(52) U.S. Cl. CPC A61B 5/02405; A61B 5/7221; A61B 5/7278

(57) ABSTRACT

An example device for determining heart rate variability (HRV) includes a memory configured to store a sensed pulse rate signal indicative of one or more sensed pulse rates and processor circuitry. The processor circuitry is configured to receive the sensed pulse rate signal and determine that a pulse rate of the sensed pulse rate signal, within a predetermined time period, is erroneous. The processor circuitry is configured to process the erroneous pulse rate to create a modified sensed pulse rate signal. The processor circuitry is configured to determine an HRV value based on the modified pulse rate signal over the predetermined time period and output information indicative of the determined HRV value.

This application claims priority to U.S. Provisional Application No. 63/250,775, filed Sep. 30, 2021, the entire contents of which is hereby incorporated by reference.

### TECHNICAL FIELD

This disclosure relates to systems, devices, and methods for determining heart rate variability.

### BACKGROUND

Oximetry may be used in the clinical setting to measure, in a non-invasive manner, blood characteristics. For example, oximetry may be used to estimate arterial blood oxygenation and sense the pulse rate of a patient, which may be a proxy for heart rate. To sense pulse rate, two optical sources, typically light-emitting-diodes (LEDs), are used to inject light into tissue of the patient. A photodiode may be used to capture the light after propagating through blood perfused tissue. During a cardiac cycle, the amount of blood in the optical path changes, which changes the amount the light that is absorbed by the photodiode. As more light is absorbed, the photodiode produces less photocurrent. Hence, during the cardiac cycle the photocurrent from the photodiode is a modulated photocurrent producing a pulsatile waveform associated with each heartbeat. The period between each beat is the pulse period. The pulse rate may be derived from this pulsatile waveform. Heart rate variability (HRV) may be determined from a sensed pulse rate.

### SUMMARY

In general, this disclosure relates to devices, systems, and techniques for determining heart rate variability (HRV). HRV is a vital sign that estimates the amount of variability in heart rate. In some examples, HRV may be measured as a moving standard deviation of a pulse rate over a defined moving time period (e.g., 5-minutes). Therefore, a device for determining HRV values, such as an oximetry device, should address (e.g., account for) invalid input pulse rate samples over the HRV moving time period. It may be further desirable to require a minimum number of valid pulse rate samples within the defined time period to improve reliability and accuracy of the determined HRV values.

In one example, a device includes a memory configured to store a sensed pulse rate signal indicative of one or more sensed pulse rates and processor circuitry configured to: receive the sensed pulse rate signal; determine that a pulse rate of the sensed pulse rate signal, within a predetermined time period, is erroneous; process the erroneous pulse rate to create a modified sensed pulse rate signal; determine an HRV value based on the modified pulse rate signal over the predetermined time period; and output information indicative of the determined HRV value.

In another example, a method includes receiving a sensed pulse rate signal indicative of one or more sensed pulse rates; determining that a pulse rate of the sensed pulse rate signal, within a predetermined time period, is erroneous; processing the erroneous pulse rate to create a modified sensed pulse rate signal; determining an HRV value based on the modified pulse rate signal over the predetermined time period; and outputting information indicative of the determined HRV value.

In one example, a non-transitory computer-readable storage medium includes instructions, which, when executed, cause processor circuitry to receive a sensed pulse rate signal indicative of one or more sensed pulse rates; determine that a pulse rate of the sensed pulse rate signal, within a predetermined time period, is erroneous; process the erroneous pulse rate to create a modified sensed pulse rate signal; determine a heart rate variability (HRV) value based on the modified pulse rate signal over the predetermined time period; and output information indicative of the determined HRV value.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is a conceptual block diagram illustrating an example oximetry device.

FIG. 2 is a conceptual block diagram illustrating an example oximetry device configured to monitor heart rate variability of a patient.

FIG. 3 is a flow diagram illustrating example multiple rate filtering techniques of this disclosure.

FIG. 4 is a graphical diagram depicting an example use of multiple rate qualifications when determining an HRV value.

FIG. 5 is a graphical diagram depicting example dropout techniques according to this disclosure.

FIG. 6 is a flow diagram illustrating an example technique for determining and outputting an HRV value, in accordance with the techniques of this disclosure.

FIG. 7 is a flow diagram illustrating another example technique for determining and outputting an HRV value, in accordance with the techniques of this disclosure.

### DETAILED DESCRIPTION

As mentioned above, heart rate variability (HRV) may be determined as a standard deviation of a pulse rate signal over a moving predetermined time period, such as 5 minutes. As an HRV determination relies on a sensed pulse rate, any erroneous pulse rate data may cause an effect that will last for the same time period used for determining the HRV value, for example, 5 minutes. This error propagation in time may be especially impactful in the case of: 1) erroneous multiplying due to inaccurate or erroneous detection of two pulses instead of one (e.g., doubling) or inaccurate or erroneous detection of one pulse instead of two (e.g., halving) of pulse rate; 2) pulse rate dropouts followed by an excessive difference in resumed pulse rate when compared to the last pulse rate value before the dropout; 3) long periods of holding a pulse rate value may cause artificially reduced HRV values that might trigger undesired HRV related alarms; 4) potential erroneous pulse rate supported by additional input signals; or 5) insufficient reliable pulse rate samples. A reduction in heart rate variability (HRV) has been connected to clinical outcomes such as sepsis and intraventricular hemorrhage, among others. Therefore, it may be desirable for systems, devices, or methods to address (e.g., account for) any problems in a sensed pulse rate signal prior to determining an HRV value so as to increase the accuracy of determined HRV values.

An oximeter is a medical device configured to determine an oxygen saturation of an analyzed tissue. An oximeter may also be configured to sense a pulse rate and HRV of a person, such as a patient. An oximeter may also measure other characteristics and chemical compositions of blood.

An oximeter may include a sensor device that is placed at a site on a patient, for example, on a fingertip, toe, forehead or earlobe, the cerebral cortex, or in the case of a neonate, across a foot, across a hand, or another location. The oximeter may use a light source to pass light through blood perfused tissue and photoelectrically sense the absorption of the light in the tissue. Additional suitable sensor locations may include, for example, a neck to monitor carotid artery pulsatile flow, a wrist to monitor radial artery pulsatile flow, an inside of a patient’s thigh to monitor femoral artery pulsatile flow, an ankle to monitor tibial artery pulsatile flow, around or in front of an ear, locations with strong pulsatile arterial flow, or other locations.

The oximeter may be configured to output a photonic signal that interacts with tissue at one or more wavelengths that are attenuated by the blood in an amount representative of the blood constituent concentration. The oximeter may be configured to generate the photonic signal at red and infrared (IR) wavelengths. The oximeter may estimate the blood oxygen saturation of hemoglobin in arterial blood based on an intensity of the photonic signal at the red wavelength and the photonic signal at the infrared wavelength.

Light emitting diodes (LEDs) of an oximeter may be manufactured to output a photonic signal at a particular wavelength with a manufacturing tolerance. For example, a first LED may output a first phonic signal (e.g., red light) at a first wavelength range (e.g., 630 nm -700 nm) with a first manufacturing tolerance of 5 %. In this example, a second LED may output a second phonic signal (e.g., infrared light) at a second wavelength range (e.g., 700 nm - 1200 nm) with a second manufacturing tolerance of 5 %. While various examples described herein refer to a LED that may output relatively low intensity light, in some examples, LEDs may include devices that output relatively intense beams of light of infrared radiation (e.g., laser diodes), vertical-cavity surface-emitting laser, or another device that emits light using at least one p-type junction and at least one n-type junction. Moreover, while examples described herein may refer to a device emitting light (e.g., LED, laser diode, etc.) similar techniques may be used with devices that receive light (e.g., photodiodes).

In accordance with the techniques of the disclosure, a device (e.g., an oximeter) may be configured to receive a sensed pulse rate signal and determine that a received sensed pulse rate of the sensed pulse rate signal, within a predetermined time period, is erroneous. The device may be configured to process the erroneous received sensed pulse rate to create a modified sensed pulse rate signal. The device may be configured to determine an HRV value based on the modified pulse rate signal over the predetermined time period and output information indicative of the determined HRV value. In this way, the example techniques provide a technical solution to a technical problem related to possible errors in determining HRV, and incorporate the techniques into a practical application for determining HRV.

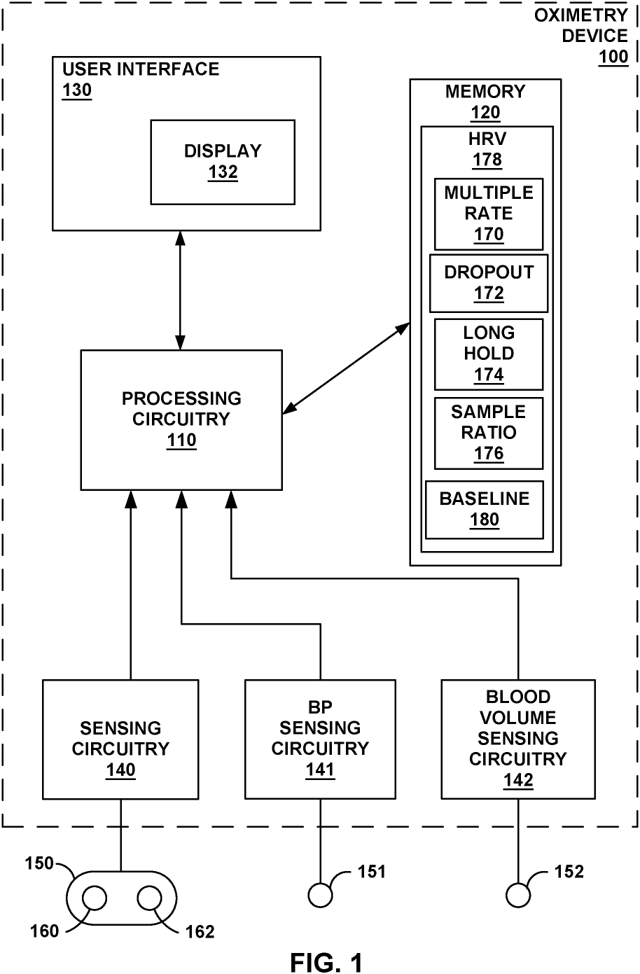

FIG. 1 is a conceptual block diagram illustrating an example oximetry device 100. While the example of FIG. 1 describes an oximetry device 100, techniques described herein for validating light emitting diodes may be used in other devices, such as, for example, a pulse oximetry device, a co-oximeter device, or another oximeter device. Oximetry device 100 includes processing circuitry 110, memory 120, user interface 130, display 132, sensing circuitry 140, 141, and 142, and sensing device(s) 150, 151, and 152. In some examples, oximetry device 100 may be configured to determine and display HRV values, e.g., during a medical procedure or for more long-term monitoring, such as monitoring of prenatal infants, children, or adults. A clinician may receive information regarding HRV values of a patient via display 132 and adjust treatment or therapy to the patient based on the HRV information. Although oximetry device 100 is described as an example device herein, other devices may determine HRV values according to the techniques of this disclosure.

Processing circuitry 110 as well as other processors, processing circuitry, controllers, control circuitry, and the like, described herein, may include one or more processors. Processing circuitry 110 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 110 may include multiple components, such as any combination of one or more microprocessors, one or more DSPs, one or more ASICs, or one or more FPGAs, as well as other discrete or integrated logic circuitry, and/or analog circuitry.

Memory 120 may be configured to store measurements of HRV values, blood pressure, oxygen saturation, blood volume, other physiological parameters, relationships between blood pressure and physiological parameters, MAP (mean arterial pressure) values, rSO2 values, COx values, BVS values, and/or HVx values, for example. Memory 120 may also be configured to store data such as thresholds used for determination of HRV values, or other thresholds for detecting abrupt changes in blood pressure, previous LLA and ULA values, and/or other physiological parameters and expected values of physiological parameters. Memory 120 may also be configured to store data such as threshold levels for physiological parameters, threshold values for blood pressure, and/or threshold levels for signal quality metrics. The thresholds or other data may stay constant throughout the use of device 100 and across multiple patients, or these values may change over time.

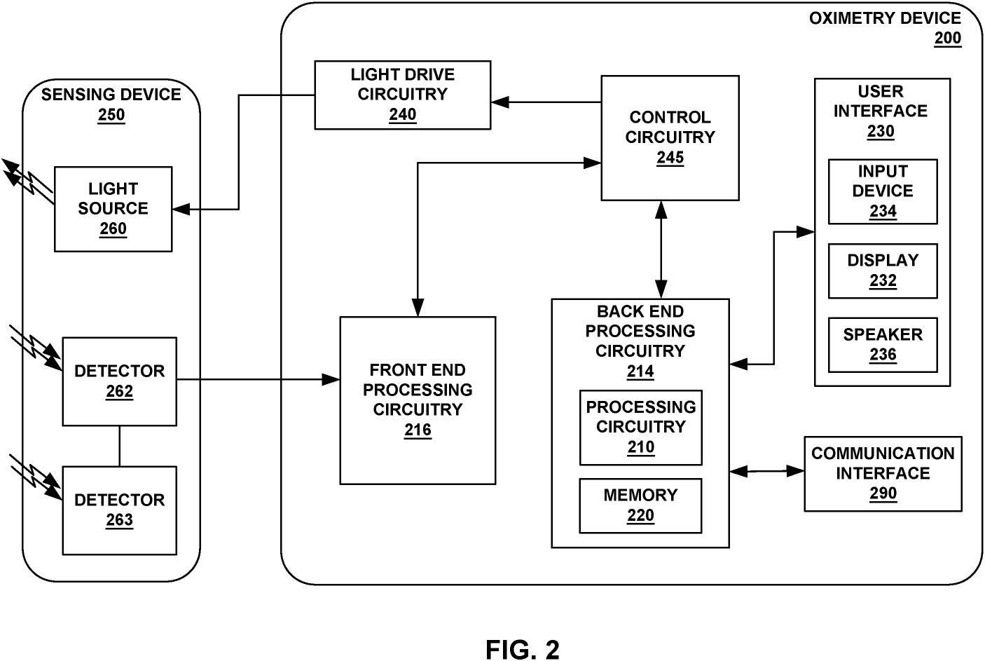

Memory 120 may store program instructions, which may include one or more program modules, which are executable by processing circuitry 110. When executed by processing circuitry 110, such program instructions may cause processing circuitry 110 to provide the functionality ascribed to it herein. For example, memory 120 may store instructions regarding how to determine erroneous pulse rate values, whether sufficient valid pulse rate samples have been sensed to determine a relatively accurate HRV, abrupt changes in measured blood pressure, calculating ULA and LLA values, and presenting information to the user via user interface 130. The program instructions may be embodied in software, and/or firmware. Memory 120, as well as other memory devices described herein (e.g., memory 220 shown in FIG. 2), may include any volatile, non-volatile, magnetic, optical, circuitry, or electrical media, such as a random access memory (RAM), read-only memory (ROM), non-volatile RAM (NVRAM), electrically-erasable programmable ROM (EEPROM), flash memory, or any other digital media.

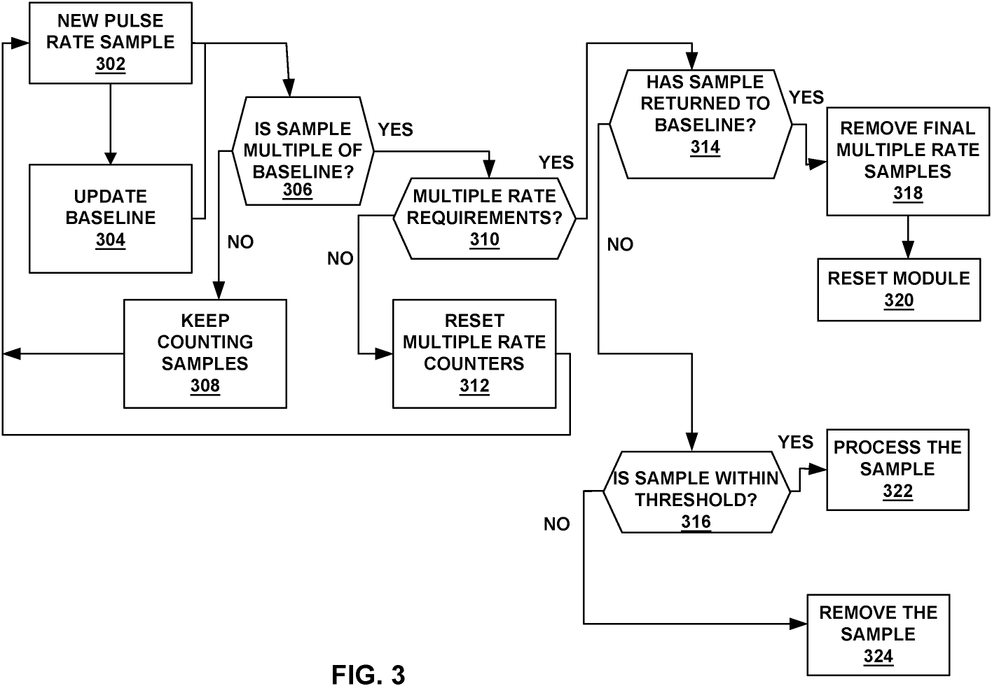

For example, memory 120 may also include HRV module 178 which may include program instructions which may cause processing circuitry 110 to determine an HRV value based on a sensed pulse rate signal. In some examples, HRV module 178 may include multiple rate module 120 which may include program instructions which may cause processing circuitry 110 to determine whether a sensed pulse rate sample is approximately (e.g., within a predetermined multiple rate threshold of) a multiple of a baseline pulse rate and, based on the sensed pulse rate sample being approximately a multiple of the baseline pulse rate, then remove or modify that sensed pulse rate sample from a sensed pulse rate signal. HRV module 178 may also include dropout module 172 which may include program instructions which may cause processing circuitry 110 to determine whether a sensed pulse rate sample is a dropout sample followed by an excessive difference in resumed pulse rate when compared to the last pulse rate value before the dropout and remove the resumed pulse rate sample from the sensed pulse rate signal. HRV module 178 may also include long hold module 174 which may include program instructions which may cause processing circuitry 110 to determine whether a sensed pulse rate sample is a part of a long hold and remove the long hold sample from the sensed pulse rate signal. HRV module 178 may also include sample ratio module 176 which may include program instructions which may cause processing circuitry 110 to determine whether a sufficient ratio of valid pulse rate samples have been sensed and not including an HRV value if a sufficient ratio of valid pulse rate sample have not been sensed in an HRV value being output. HRV module 178 may also include baseline module 180 which may include program instructions which may cause processing circuitry 110 to determine a baseline pulse rate based on a sensed pulse rate signal. While certain modules are depicted in FIG. 1, in some examples there may be fewer or more modules than those that are illustrated, and, in some examples, not all modules are needed. The functioning of these modules is discussed in more detail below and with respect to FIGS. 3, 6, and 7.

User interface 130 and/or display 132 may be configured to present information to a user (e.g., a clinician). User interface 130 and/or display 132 may be configured to present a graphical user interface to a user, where each graphical user interface may include indications of values of one or more physiological parameters of a subject. For example, processing circuitry 110 may be configured to present HRV values, pulse rate values, blood pressure values, or other physiological parameter values (e.g., heart rate), of a patient via display 132. User interface 130 and/or display 132 may include a monitor, cathode ray tube display, a flat panel display such as a liquid crystal (LCD) display, a plasma display, or a light emitting diode (LED) display, personal digital assistant, mobile phone, tablet computer, laptop computer, any other suitable display device, or any combination thereof. User interface 130 may also include means for projecting audio to a user, such as speaker(s). Processing circuitry 110 may be configured to present, via user interface 130, a visual, audible, or somatosensory notification (e.g., an alarm signal) indicative of the patient’s HRV, or pulse rate. User interface 130 may include or be part of any suitable device for conveying such information, including a computer workstation, a server, a desktop, a notebook, a laptop, a handheld computer, a mobile device, or the like. In some examples, processing circuitry 110 and user interface 130 may be part of the same device or supported within one housing (e.g., a computer or monitor).

Sensing circuitry 140, 141, and 142 may be configured to receive physiological signals sensed by respective sensing device(s) 150, 151, and 152 and communicate the physiological signals to processing circuitry 110. Sensing device(s) 150, 151, and 152 may include any sensing hardware configured to sense a physiological parameter of a patient, such as, but not limited to, one or more electrodes, optical receivers, blood pressure cuffs, or the like. Sensing circuitry 140, 141, and 142 may convert the physiological signals to usable signals for processing circuitry 110, such that processing circuitry 110 is configured to receive signals generated by sensing circuitry 140, 141, and 142. Sensing circuitry 140, 141, and 142 may receive signals indicating physiological parameters from a patient, such as, but not limited to, pulse rate, blood pressure, oxygen saturation, heart rate, and respiration. Sensing circuitry 140, 141, and 142 may include, but are not limited to, pulse rate, blood pressure sensing circuitry, oxygen saturation sensing circuitry, heart rate sensing circuitry, temperature sensing circuitry, electrocardiography (ECG) sensing circuitry, electroencephalogram (EEG) sensing circuitry, or any combination thereof. In some examples, sensing circuitry 140, 141, and 142 and/or processing circuitry 110 may include signal processing circuitry such as an analog-to-digital converter. In some examples, oximetry device 100 may not include sensing circuitry 141 sensing circuitry 142, sensing device 151, or sensing device 152.

Oxygen saturation sensing device 150 is an oxygen saturation sensor configured to generate an oxygen saturation signal indicative of blood oxygen saturation within the venous, arterial, and/or capillary systems within a region of the patient. For example, oxygen saturation sensing device 150 may be configured to be placed on the patient’s forehead and may be used to determine the oxygen saturation of the patient’s blood within the venous, arterial, and/or capillary systems of a region underlying the patient’s forehead (e.g., in the cerebral cortex).

Oxygen saturation sensing device 150 may include emitter 160 and detector 162. In some examples, emitter 160 may include at least two light emitting diodes (LEDs), each configured to emit at different wavelengths of light, e.g., red and near infrared light. In some examples, light drive circuitry (e.g., within sensing device 150, sensing circuitry 140, and/or processing circuitry 110) may provide a light drive signal to drive emitter 160 and to cause emitter 160 to emit light. In some examples, the LEDs of emitter 160 emit light in the wavelength range of about 600 nanometers (nm) to about 1000 nm. In a particular example, one LED of emitter 160 is configured to emit light at a wavelength of about 730 nm and the other LED of emitter 160 is configured to emit light at a wavelength of about 810 nm. Other wavelengths of light may also be used in other examples.

In some examples, detector 162 may include a detection element configured to sense a light intensity in tissue of a person that is generated by emitter 160. If two wavelengths are used, the two wavelengths may be contrasted to arrive at an oxygen saturation value. Oxygen saturation sensing device 150 may provide the oxygen saturation signal to processing circuitry 110 or to any other suitable processing device.

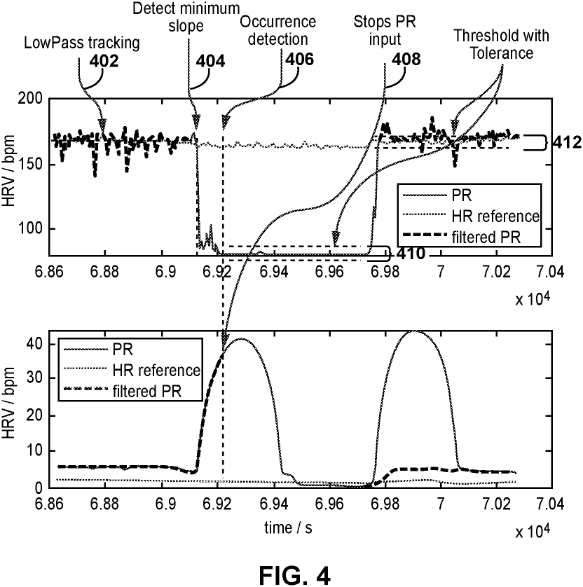

In some examples, processing circuitry 110 may determine and output an HRV value in real-time while processing incoming pulse rate samples, for example by executing HRV module 178. In some examples, processing circuitry 110 may delay output of an HRV value to allow oximetry device 100 to qualify any problems detected in a defined backward time window. Because HRV is normally considered a long-term trend metric, a relatively small time delay (e.g., 30 secs, 1 minute, 2 minutes, or the like) would likely be acceptable to most clinicians. For example, by delaying the output of the HRV value by the relatively small time delay, processing circuitry 110 may remove or correct erroneous pulse rates, such as multiple rate occurrences, dropouts, and pulse rate long holds, prior to outputting a determined HRV value. For example, in this manner, processing circuitry 110 may remove the initial slope in HRV (such as is depicted in FIG. 4) which may occur prior to detection.

In some examples, there may be rare events where an input pulse rate could potentially be in error and it may be desirable for processing circuitry 110 to remove the erroneous input pulse rates prior to completing the standard deviation calculation used to determine the HRV value. For example, an input pulse rate may be a dropout pulse rate (e.g., no pulse rate) followed by an excessive difference in resumed pulse rate when compared to the last pulse rate value before the dropout due to movement or removal and reattachment of a pulse rate sensor, a multiple pulse rate due to erroneous detection of two pulses instead of one (e.g., doubling) or erroneous detection of one pulse instead of two (e.g., halving), a long-hold pulse rate due to movement of the patient, or the like.

For example, oximetry device 100 may utilize HRV module 178 that incorporates other modules (e.g., multiple rate module 170, dropout module 172, long hold module 174, and/or sample ratio module 176) that address problems described herein in an independent manner such that one or more of the potential problems described in this disclosure may be addressed, according to the techniques of this disclosure.

One potential problem is having a sufficient samples-to-dropout ratio. In some examples, processing circuitry 110 may determine an HRV value by using a 5-minute moving standard deviation, or other suitable time period, of a sensed pulse rate or sensed heart rate (e.g., derived from an ECG monitor). In order to validate any given period over which the standard deviation is calculated, processing circuitry 110 executing sample ratio module 176 may require a minimum ratio of valid samples-to-dropouts or valid samples-to-overall samples (e.g., 90% of the time period should contain valid samples).

In some examples, oximetry device 100 may calculate a ratio of sufficient samples-to-dropouts (or valid samples-to-overall samples) after interpolating the input pulse rate to a fixed sample rate. For example, 90% of 300 samples in a 5-minute interval, at a sample rate of 1 Hz.

Another potential problem is the input pulse rate suddenly changing to a multiple (such as double or half) of the pulse rate at an immediately previous time period or a baseline pulse rate due to erroneous detection of two pulses instead of one (e.g., doubling) or one pulse instead of two (e.g., halving). For example, an input pulse rate may double or half and processing circuitry 110 executing multiple rate module 170 may be configured to detect cases where the input pulse rate suddenly changes to double or half the pulse rate compared to an immediately previous time period or the baseline pulse rate. If oximetry device 100 detects such a condition, oximetry device 100 may attempt to correct such values, remove such values from the sensed pulse rate signal, or stop reporting HRV values during such an event. Oximetry device 100 may then resume outputting the HRV determination when oximetry device 100 detects the pulse rate is back within the expected range (e.g., within a predetermined range of a previous pulse rate or baseline pulse rate), or after a timeout period has elapsed. This timeout period may be equal to, greater to, or less than the time period used to calculate the moving standard deviation (e.g., 5 minutes).

In some examples, processing circuitry 110 executing long hold module 174 may stop accepting sensed pulse rate values (or remove such values from a sensed pulse rate signal) when the input pulse rate values are held constant or less than a numerical tolerance, e.g., 0.01 beats-per-minute (bpm), beyond a predetermined constant time limit threshold. For example, a pulse rate value may be held constant during prolonged periods of a signal artifact, for example. For example, if a device is unable to qualify new pulses when determining pulse rate, the device may hold the pulse rate for a short period of time, rather than immediately dropping out. Processing circuitry 110 may resume accepting pulse rate samples when processing circuitry 110 detects a change in sensed pulse rate value.

For example, processing circuitry 110 may stop accepting sensed pulse rate samples (or remove such values from the sensed pulse rate signal) beyond 30 seconds of a held pulse rate value. This predetermined constant time limit threshold can be dynamically, or statically, changed to other values, e.g., 10 seconds or 60 seconds, depending on rate source characteristics - for example of an ECG monitor.

In some examples, long hold module 174 may include a pulse oximeter algorithm metric (RateAge) that tracks the held rate age in a relatively sophisticated manner. For example, using RateAge, processing circuitry 110 may down weight samples with a large Rate Age (e.g., pulse rates held for longer) in the standard deviation calculation for HRV rather than causing the HRV signal to stop. This may improve HRV uptime (e.g., the ratio of time HRV is posting in relation to a time the input pulse rate is posting) during long holds in the sensed pulse rate signal.

Another potential problem is signal integrity. It may be desirable to remove values from the moving time window used to determine the HRV value where there is a high likelihood the sensed pulse rate may be in error. This could occur, for example, during periods of prolonged excessive signal artifact(s). Processing circuitry 110 may detect such a high likelihood that a pulse rate may be in error by using additional input quality metrics such as percent modulation, a motion flag, ratio of ratios (RoR) variance, signal to noise ratio, or some other signal quality metric currently available from oximetry device 100 (such as a pulse oximeter). For example, percent modulation, which is the modulation amplitude (in percentage regarding the absolute signal) in one or more of the oximeter LED signals, correlates positively with a signal artifact and a threshold may be set above which the pulse rate samples are removed. In another example, weights might be attributed to the pulse rate samples depending on corresponding percent modulation values, reducing the impact of high percent modulation samples. Processing circuitry 110 may remove or down weight such input samples in the standard deviation calculation of HRV. As such, oximetry device 100 may make the HRV calculation more robust to noise or other artifacts.

In some examples, processing circuitry 110 may employ outlier detection techniques to remove spurious pulse rate samples without an additional “integrity” signal. For example, oximetry device 100 may remove pulse rate samples whose values are more than a predetermined difference threshold from a previous pulse rate value or a determined baseline pulse rate value (for example, determined by executing baseline module 180).

In some examples, instead of or, in addition to using pulse rate to determine HRV, oximetry device 100 may use other measurements of or surrogates for heart rate, such as one or more signals from an ECG monitor, an arterial blood pressure monitor, or a ballistocardiogram.

Blood pressure sensing device 151 and oxygen saturation sensing device 150 may each be placed on the same or different parts of the patient’s body. For example, blood pressure sensing device 151 and oxygen saturation sensing device 150 may be physically separate from each other and separately placed on the patient. As another example, blood pressure sensing device 151 and oxygen saturation sensing device 150 may in some cases be part of the same sensor or supported by a single sensor housing. For example, blood pressure sensing device 151 and oxygen saturation sensing device 150 may be part of an integrated oximetry system configured to non-invasively measure blood pressure (e.g., based on time delays in a PPG signal) and oxygen saturation. One or both of blood pressure sensing device 151 or oxygen saturation sensing device 150 may be further configured to measure other parameters, such as HRV, pulse rate, hemoglobin, respiratory rate, respiratory effort, heart rate, saturation pattern detection, response to stimulus such as bispectral index (BIS) or electromyography (EMG) response to electrical stimulus, or the like. While an example oximetry device 100 is shown in FIG. 1, the components illustrated in FIG. 1 are not intended to be limiting. Additional or alternative components and/or implementations may be used in other examples.

For example, to sense a pulse rate, oximetry device 100 may use emitter 160 to inject light into tissue of the patient. Detector 162 may capture the light after the light propagates through blood perfused tissue. During a cardiac cycle, the amount of blood in the optical path changes which changes the amount the light that is absorbed by detector 162. As more light is absorbed, detector 162 produces less photocurrent. Hence, during the cardiac cycle the photocurrent from detector 162 is a modulated photocurrent producing a pulsatile waveform associated with each heartbeat. The period between each beat is the pulse period. The pulse rate may be derived from this pulsatile waveform. Heart rate variability (HRV) may be determined from a sensed pulse rate.

Blood pressure sensing device 151 may be any sensor or device configured to obtain the patient’s blood pressure (e.g., arterial blood pressure). In one example, the blood pressure sensing device 151 may include or be connected to a probe configured to be inserted into a blood pressure of the patient. In another example, blood pressure sensing device 151 may include a blood pressure cuff for non-invasively monitoring blood pressure or an arterial line for invasively monitoring blood pressure (e.g., a pressure probe configured to be placed within an artery or vein). In certain examples, blood pressure sensing device 151 may include one or more pulse oximetry sensors. In some such cases, the patient’s blood pressure may be derived by processing time delays between two or more characteristic points within a single plethysmography (PPG) signal obtained from a single pulse oximetry sensor.

Processing circuitry 110 may be configured to receive one or more physiological signals generated by sensing devices 150, 151, and 152 and sensing circuitry 140, 141, and 142. The physiological signals may include a signal indicating pulse rate, HRV, blood pressure, a signal indicating oxygen saturation, and/or a signal indicating blood volume of a patient. Processing circuitry 110 may be configured to determine a relationship between blood pressure values of the patient and a physiological parameter of the patient, such as a correlation index (e.g., COx, a hemoglobin volume index (HVx)), an oxygen saturation value, a blood volume value, a gradient-based metric of two or more physiological parameters, and/or another physiological parameter. Processing circuitry 110 can determine a gradients-based metric by determining respective gradients of signals for physiological parameters and determining whether the respective gradients trend together.

In the above examples, processing circuitry 110, and light drive circuitry (e.g., within sensing device 150, sensing circuitry 140, and/or processing circuitry 110), are described as performing the example techniques, wherein light drive circuitry may be part of processing circuitry 110, sensing device 150 and/or sensing circuitry 140). However, any one or combination of processing circuitry 110, sensing circuitry 140, and/or sensing device 150 may be configured to perform the example techniques. For instance, the example techniques may be performed by circuitry, and example of the circuitry includes any one or any combination of processing circuitry 110, sensing circuitry 140, and/or sensing device 150.

FIG. 2 is a conceptual block diagram illustrating an example oximetry device 200 configured to monitor the HRV of a patient. In the example shown in FIG. 2, oximetry device 200 is coupled to sensing device 250 and may be collectively referred to as an oximetry system, which each generate and process physiological signals of a subject. Oximetry device 200 and sensing device 250 may be examples of oximetry device 100 and sensing device 150, respectively, of FIG. 1. In some examples, sensing device 250 and oximetry device 200 may be part of an oximeter. As shown in FIG. 2, oximetry device 200 includes back-end processing circuitry 214, user interface 230, light drive circuitry 240, front-end processing circuitry 216, control circuitry 245, and communication interface 290. Oximetry device 200 may be communicatively coupled to sensing device 250. Oximetry device 200 is an example of oximetry device 100 shown in FIG. 1. In some examples, oximetry device 200 may also include a blood pressure sensor and/or a blood volume sensor (e.g., sensing devices 151 and 152 of FIG. 1).

In the example shown in FIG. 2, sensing device 250 includes light source 260, detector 262, and detector 263. Light source 260 may be an example of emitter 160 of FIG. 1. Detectors 262 and 263 may be examples of detector 162 of FIG. 1. In some examples, sensing device 250 may include a single detector or more than two detectors. Light source 260 may be configured to emit photonic signals having two or more wavelengths (e.g., up to four or more wavelengths, more than 4 wavelengths, etc.) of light (e.g., red and infrared (IR), or another wavelength of light) into a subject’s tissue. For example, light source 260 may include a red light emitting light source and an IR light emitting light source, (e.g., red and IR LEDs), for emitting light into the tissue of a subject to generate physiological signals. In some examples, the red wavelength may be between about 600 nm and about 700 nm, and the IR wavelength may be between about 800 nm and about 1000 nm. Other wavelengths of light may be used in other examples. Light source 260 may include any number of light sources with any suitable characteristics. In examples in which an array of sensors is used in place of sensing device 250, each sensing device may be configured to emit a single wavelength. For example, a first sensing device may emit only a red light while a second sensing device may emit only an IR light. In some examples, light source 260 may be configured to emit two or more wavelengths of near-infrared light (e.g., wavelengths between 600 nm and 1000 nm) into a subject’s tissue. In some examples, light source 260 may be configured to emit four wavelengths of light (e.g., 724 nm, 770 nm, 810 nm, and 850 nm) into a subject’s tissue. In some examples, the subject may be a medical patient.

As used herein, the term “light” may refer to energy produced by radiative sources and may include one or more of ultrasound, radio, microwave, millimeter wave, infrared, visible, ultraviolet, gamma ray or X-ray electromagnetic radiation. Light may also include any wavelength within the radio, microwave, infrared, visible, ultraviolet, or X-ray spectra, and that any suitable wavelength of electromagnetic radiation may be appropriate for use with the present techniques. Detectors 262 and 263 may be chosen to be specifically sensitive to the chosen targeted energy spectrum of light source 260.

Detectors 262 and 263 may be configured to detect the intensity of multiple wavelengths of near-infrared light. In some examples, detectors 262 and 263 may be configured to detect the intensity of light at the red and IR wavelengths. In some examples, an array of detectors may be used and each detector in the array may be configured to detect an intensity of a single wavelength. In operation, light may enter detector 262 after passing through the subject’s tissue, including skin, bone, and other shallow tissue (e.g., non-cerebral tissue and shallow cerebral tissue). Light may enter detector 263 after passing through the subject’s tissue, including skin, bone, other shallow tissue (e.g., non-cerebral tissue and shallow cerebral tissue), and deep tissue (e.g., deep cerebral tissue). Detectors 262 and 263 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detectors 262 and 263.

For example, detectors 262 and/or detector 263 may determine a first intensity of a first received photonic signal corresponding to a first output photonic signal (e.g., red light) output using a first light emitting diode of light source 260. More specifically, processing circuitry (e.g., light drive circuitry 240) may be configured to drive the first light emitting diode of light source 260 to output the first output photonic signal towards a subject’s tissue and receive, from detector 262 and/or detector 263, the first received photonic signal after the first output photonic signal transmits through the subject’s tissue. Similarly, detectors 262 and/or detector 263 may determine a second intensity of a second received photonic signal corresponding to a second output photonic signal (e.g., infrared light) output using the second light emitting diode. More specifically, processing circuitry (e.g., light drive circuitry 240) may be configured to drive a second light emitting diode of light source 260 to output the second output photonic signal towards the subject’s tissue and receive, from detector 262 and/or detector 263, the second received photonic signal after the second output photonic signal transmits through the subject’s tissue.

After converting the received light to an electrical signal, detectors 262 and 263 may send the detection signals to oximetry device 200, which may process the detection signals and determine physiological parameters (e.g., based on the absorption of the red and IR wavelengths in the subject’s tissue at both detectors). For example, oximetry device 200 may determine a pulse rate, HRV, and/or an oxygen saturation level based on the first intensity of the first received photonic signal and the second intensity of the second received photonic signal.

Processing circuitry 210 may output information indicative of an HRV value. For example, processing circuitry 210 may remove erroneous pulse rate samples or modify erroneous pulse rate samples to create a modified pulse rate signal. Processing circuitry 210 may also not include samples in the modified pulse rate signal when there are insufficient valid samples to warrant including the samples in a determination of an HRV value. Processing circuitry 210 may then determine an HRV value by determining a standard deviation of the modified pulse rate signal over a predetermined time period. Processing circuitry 210 may output the HRV value to display 232 and/or store an indication of the HRV value (e.g., a numerical value indicating the HRV) in memory 220.

Processing circuitry 210 may determine the oxygen saturation level based on a first wavelength for the first output photonic signal and a second wavelength for the second output photonic signal. For instance, processing circuitry 210 may determine the oxygen saturation level by matching an amount of absorption of the first wavelength (e.g., a difference in magnitude between an emitted light and a received light) and matching an amount of absorption of the second wavelength in a table and outputting a corresponding oxygen saturation level for the absorption of the first wavelength and the absorption of the second wavelength.

Processing circuitry 210 may output an indication of the oxygen saturation level. For example, processing circuitry 210 may store an indication of the oxygen saturation level (e.g., a numerical value indicating the oxygen saturation level) for storage at memory 220. Processing circuitry 210 may output an indication of the oxygen saturation level (e.g., a numerical value indicating the oxygen saturation level) to user interface 230 for output on display 232. Processing circuitry 210 may output an indication of the oxygen saturation level (e.g., a numerical value indicating the oxygen saturation level) to communication interface 290 for storage and/or output at one or more external or implanted devices.

Control circuitry 245 may be coupled to light drive circuitry 240, front-end processing circuitry 216, and back-end processing circuitry 214, and may be configured to control the operation of these components. In some examples, control circuitry 245 may be configured to provide timing control signals to coordinate their operation. For example, light drive circuitry 240 may generate one or more light drive signals, which may be used to turn on and off light source 260, based on the timing control signals provided by control circuitry 245. Front-end processing circuitry 216 may use the timing control signals to operate synchronously with light drive circuitry 240. For example, front-end processing circuitry 216 may synchronize the operation of an analog-to-digital converter and a demultiplexer with the light drive signal based on the timing control signals. In addition, the back-end processing circuitry 214 may use the timing control signals to coordinate its operation with front-end processing circuitry 216.

Light drive circuitry 240, as discussed above, may be configured to generate a light drive signal that is provided to light source 260 of sensing device 250. The light drive signal may, for example, control the intensity of light source 260 and the timing of when light source 260 is turned on and off. In some examples, light drive circuitry 240 provides one or more light drive signals to light source 260. Where light source 260 is configured to emit two or more wavelengths of light, the light drive signal may be configured to control the operation of each wavelength of light. The light drive signal may comprise a single signal or may comprise multiple signals (e.g., one signal for each wavelength of light).

Front-end processing circuitry 216 may perform any suitable analog conditioning of the detector signals. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, front-end processing circuitry may determine a baseline pulse rate by low pass filtering a sensed pulse rate signal. The conditioned analog signals may be processed by an analog-to-digital converter of circuitry 216, which may convert the conditioned analog signals into digital signals. Front-end processing circuitry 216 may operate on the analog or digital form of the detector signals to separate out different components of the signals. Front-end processing circuitry 216 may also perform any suitable digital conditioning of the detector signals, such as low pass, high pass, band pass, notch, averaging, or any other suitable filtering, amplifying, performing an operation on the signal, performing any other suitable digital conditioning, or any combination thereof. Front-end processing circuitry 216 may decrease the number of samples in the digital detector signals. In some examples, front-end processing circuitry 216 may also remove dark or ambient contributions to the received signal.

Back-end processing circuitry 214 may include processing circuitry 210 and memory 220. Processing circuitry 210 may include an assembly of analog or digital electronic components and may be configured to execute software, which may include an operating system and one or more applications, as part of performing the functions described herein with respect to, e.g., processing circuitry 110 of FIG. 1. Processing circuitry 210 may receive and further process physiological signals received from front-end processing circuitry 216. For example, processing circuitry 210 may determine one or more physiological parameter values based on the received physiological signals. For example, processing circuitry 210 may compute one or more of blood oxygen saturation (e.g., arterial, venous, or both), pulse rate, HRV, respiration rate, respiration effort, blood pressure, hemoglobin concentration (e.g., oxygenated, deoxygenated, and/or total), any other suitable physiological parameters, or any combination thereof.

Processing circuitry 210 may perform any suitable signal processing of a signal, such as any suitable band-pass filtering, adaptive filtering, closed-loop filtering, any other suitable filtering, and/or any combination thereof. Processing circuitry 210 may also receive input signals from additional sources not shown. For example, processing circuitry 210 may receive an input signal containing information about treatments provided to the subject from user interface 230. Additional input signals may be used by processing circuitry 210 in any of the determinations or operations it performs in accordance with back-end processing circuitry 214 or oximetry device 200.

Processing circuitry 210 is an example of processing circuitry 110 and is configured to perform the techniques of this disclosure. For example, processing circuitry 210 may be configured to receive a sensed pulse rate signal and determine that a received sensed pulse rate of the sensed pulse rate signal, within a predetermined time period, is erroneous. Processing circuitry 210 may be configured to process the erroneous received sensed pulse rate to create a modified sensed pulse rate signal. For example, processing circuitry 210 may drop or delete the erroneous received sensed pulse rate, replace the erroneous sensed pulse rate with a previously sensed pulse rate or baseline pulse rate, interpolate a pulse rate, or the like. Processing circuitry 210 may be configured to determine an HRV based on the modified pulse rate signal over the predetermined time period and output information indicative of the determined HRV via user interface 230 (e.g., through display 232).

Memory 220 may include any suitable computer-readable media capable of storing information that can be interpreted by processing circuitry 210. In some examples, memory 220 may store reference absorption curves, reference sets, determined values, such as pulse rate, HRV, blood oxygen saturation, pulse rate, blood pressure, fiducial point locations or characteristics, initialization parameters, any other determined values, or any combination thereof, in a memory device for later retrieval. Memory 220 may also store thresholds for determining an HRV value, detecting abrupt changes in blood pressure, and so on. Back-end processing circuitry 214 may be communicatively coupled with user interface 230 and communication interface 290.

User interface 230 may include input device 234, display 232, and speaker 236 in some examples. User interface 230 is an example of user interface 130 shown in FIG. 1, and display 232 is an example of display 132 shown in FIG. 1. User interface 230 may include, for example, any suitable device such as one or more medical devices (e.g., a medical monitor that displays various physiological parameters, a medical alarm, or any other suitable medical device that either displays physiological parameters or uses the output of back-end processing 214 as an input), one or more display devices (e.g., monitor, personal digital assistant (PDA), mobile phone, tablet computer, clinician workstation, any other suitable display device, or any combination thereof), one or more audio devices, one or more memory devices, one or more printing devices, any other suitable output device, or any combination thereof.

Input device 234 may include one or more of any type of user input device such as a keyboard, a mouse, a touch screen, buttons, switches, a microphone, a joystick, a touch pad, or any other suitable input device or combination of input devices. In other examples, input device 234 may be a pressure-sensitive or presence-sensitive display that is included as part of display 232. Input device 234 may also receive inputs to select a model number of sensing device 250, blood pressure sensor 250 (FIG. 2), or blood pressure processing equipment. In some examples, processing circuitry 210 may determine the type of presentation for display 232 based on user inputs received by input device 234.

In some examples, the subject may be a medical patient and display 232 may exhibit a list of values which may generally apply to the subject, such as, for example, an HRV indicator, a pulse rate indicator, an oxygen saturation signal indicator, a blood pressure signal indicator, a COx signal indicator, or a COx value indicator. Display 232 may also be configured to present additional physiological parameter information. In some examples, user interface 230 includes speaker 236 that is configured to generate and provide an audible sound that may be used in various examples, such as for example, sounding an audible notification in the event that a patient’s physiological parameters are not within a predetermined normal range and/or in the event that processing circuitry 210 determines that sensed blood pressure values may be inaccurate due to a non-physiological reason such as due to movement of a blood pressure probe of blood pressure sensor device 151 (FIG. 1).

Communication interface 290 may enable oximetry device 200 to exchange information with other external or implanted devices. Communication interface 290 may include any suitable hardware, software, or both, which may allow oximetry device 200 to communicate with electronic circuitry, a device, a network, a server or other workstations, a display, or any combination thereof. For example, oximetry device 200 may receive MAP (or other measured blood pressure) values and/or oxygen saturation values from an external device via communication interface 290.

The components of oximetry device 200 that are shown and described as separate components are shown and described as such for illustrative purposes only. In some examples the functionality of some of the components may be combined in a single component. For example, the functionality of front-end processing circuitry 216 and back-end processing circuitry 214 may be combined in a single processor system. Additionally, in some examples the functionality of some of the components of oximetry device 200 shown and described herein may be divided over multiple components. For example, some or all of the functionality of control circuitry 245 may be performed in front-end processing circuitry 216, in back-end processing circuitry 214, or both. In other examples, the functionality of one or more of the components may be performed in a different order or may not be required. In some examples, all of the components of oximetry device 200 can be realized in processor circuitry.

In the above examples, processing circuitry 210, light drive circuitry 240, and front end processing circuitry 216, are described as performing the example techniques, wherein light drive circuitry 240, and front end processing circuitry 216 may be part of processing circuitry 210). However, any one or combination of processing circuitry 210, light drive circuitry 240, or front-end processing circuitry 216 may be configured to perform the example techniques. For instance, the example techniques may be performed by circuitry, and example of the circuitry includes any one or any combination of processing circuitry 210, light drive circuitry 240, or front-end processing circuitry 216.

FIG. 3 is a flow diagram illustrating example multiple rate filtering techniques of this disclosure. While the techniques of FIG. 3 are described with respect to oximetry device 100 of FIG. 1, the techniques may be performed by another device or combination of devices.

Oximetry device 100 may receive a new pulse rate sample (302). For example, oximetry device 100 may receive a new pulse rate sample from another device or oximetry device 100 may sense a pulse rate to receive the pulse rate sample. In some examples, oximetry device 100 may update a baseline pulse rate using the new pulse rate sample (304). For example, oximetry device 100 may use a lowpass filter to determine a long-term (e.g., 2 minutes) baseline pulse rate.

Oximetry device 100 may determine whether the new pulse rate sample is a multiple of the baseline pulse rate (306). For example, oximetry device 100 may compare the baseline pulse rate to the new pulse rate sample and determine whether the new pulse rate sample is approximately half or approximately double the baseline pulse rate. For example, oximetry device 100 may determine whether the new pulse rate sample is within a predetermined multiple rate threshold of multiple value (e.g., double, or half). If the new pulse rate sample is not approximately a multiple of the baseline pulse rate (the “NO” path from box 306), oximetry device 100 may keep counting sample pulse rates (308). If the new pulse rate sample is a multiple of the baseline pulse rate (the “YES” path from box 306), oximetry device 100 may then determine whether additional multiple rate requirements have been met (310). For example, oximetry device 100 may calculate the slope from a last baseline rate value to the first rate value, after a minimum elapsed time, within this predetermined multiple rate threshold. If the magnitude of the slope is greater than a predetermined slope threshold, oximetry device 100 may delete the slope (or the sensed values of the slope) as being an erroneous slope caused by the pulse rate invalid multiplication and stop outputting HRV values. It may be desirable for oximetry device 100 to delay outputting HRV values (e.g., a 1 minute delay) to allow time for oximetry device 100 to remove such an erroneous slope so the erroneous slope is not displayed to a clinician.

Oximetry device 100 may, from the instant the first suspected rate is detected, count a number of samples from the first suspected rate onward in order to check for the ratio of samples that are within the multiple rate threshold against one or more samples that are outside the multiple rate threshold.

If the multiple rate requirements have not been met (the “NO” path from box 310), oximetry device 100 may reset a multiple rate counter (312). If the multiple rate requirements have been met (the “YES” path from box 310), oximetry device 100 may determine whether the pulse rate sample has returned to the baseline pulse rate (314). For example, oximetry device 100 may detect a return of the pulse rate to within a predetermined range or tolerance of the original baseline pulse rate (or the pulse rate prior to the occurrence of the erroneous multiple rate). If the pulse rate sample has returned to the baseline pulse rate (the “YES” path from box 314), oximetry device 100 may remove any multiple rate samples from the sensed pulse rate signal (318), creating a modified pulse rate signal, and may reset the multiple rate module (320). For example, oximetry device 100 may reset the count of samples, the multiple rate counters, and/or the baseline pulse rate.

If the pulse rate sample has not returned to the baseline pulse rate (the “NO” path from box 314), oximetry device 100 may determine whether the sample is within a threshold value of the multiple (316). If the pulse rate sample is within the threshold value of the multiple (the “YES” path from box 316), oximetry device 100 may process the sample (322), creating a modified pulse rate signal. For example, oximetry device 100 may modify the sample, such as multiply the sensed pulse rate sample by one half if the pulse rate sample is within the threshold value of the multiple and the multiple is twice the baseline pulse rate or multiply the sensed pulse rate sample by two if the pulse rate sample is within the threshold value of the multiple and the multiple is one half the baseline pulse rate. In other words, oximetry device 100 may convert incoming pulse rate values within the multiple rate threshold (which may be referred to as “threshold samples”) to more valid values, for example, by halving (in the case of threshold samples that appear to be doubled) or doubling (in the case of threshold samples that appear to be halved) the threshold samples. By converting the threshold samples to be closer to the baseline values, oximetry device 100 may start outputting HRV values earlier as opposed to if oximetry device 100 were to simply remove such samples, in which case oximetry device 100 may wait until the time window has moved on from the removed samples.

If the pulse rate sample is not within the threshold value of the multiple (the “NO” path from box 316), oximetry device 100 may remove the sample (324) from the sensed pulse rate signal, creating a modified pulse rate signal. For example, oximetry device 100 may discard (e.g., set to invalid) an incoming pulse rate sample value that is outside the multiple rate threshold.

In some examples, oximetry device 100 may track the ratio of sample values outside the multiple rate threshold to the sample values within the threshold and stop and reset the HRV determination if the ratio drops below a minimum ratio limit, for example, if the ratio of samples within the threshold to samples outside the threshold falls below 90%.

In some examples, oximetry device 100 may employ a predetermined time limit after which oximetry device 100 may reset the HRV determination to avoid latching to a false positive. For example, if the pulse rate is incorrectly detected as double the expected rate for a prolonged period of time oximetry device 100 may reset the HRV module 178 if this predetermined time limit is exceeded. In another example, rather than reset HRV module 178, after the predetermined time limited is exceeded, oximetry device 100 may reset the multiple rate module 170 to return to a valid baseline. In other words, the likelihood of an erroneous multiple of baseline rate decreases with increased pulse rate posting time at that baseline.

FIG. 4 is a graphical diagram depicting an example use of multiple rate qualifications when determining an HRV value. In some examples, when oximetry device 100 delays outputting an HRV value, oximetry device 100 may apply multiple rate qualifications, such as from data training or from template matching. For example, oximetry device 100 may use template matching in which oximetry device 100 may compare a predetermined and stored template of a multiple rate pulse rate signal to the sensed pulse rate signal and may associate large, localized correlations between the sensed pulse rate signal and the template with a multiple rate event. In some examples, oximetry device 100 may derive this archetypal multiple rate template through ensemble averaging over a number of identified multiple rate events and store the archetypal multiple rate template in memory 132. In some examples, oximetry device 100 may employ a neural networks model or other machine learning or deep learning techniques, where labeled multiple rate events are used to train a model to detect multiple rate events. For example, oximetry device 100 may employ time frequency methods, including a wavelet transform, where the morphology of the time-frequency space may be interrogated to detect features associated with multiple rate events.

For example, oximetry device 100 may use low pass filtering or tracking 402 to determine a baseline pulse rate. Oximetry device 100 may detect a minimum slope 404 of a drop (or increase) in pulse rate or heart rate. Oximetry device 100 may detect an occurrence 406 of a multiple pulse rate or heart rate. Oximetry device 100 may stop accepting sensed pulse rate samples for determination of an HRV value 408, or may remove such sensed pulse rate samples, to create a modified pulse rate signal, and concurrently oximetry device 100 may stop outputting HRV values. FIG. 4 also depicts predetermined multiple rate threshold 410 of the multiple rate and predetermined threshold range of the baseline pulse rate 412 described above.

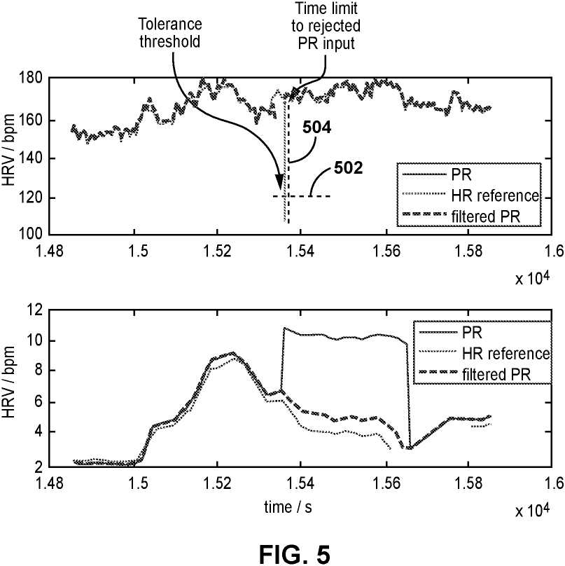

FIG. 5 is a graphical diagram depicting example dropout techniques according to this disclosure. For example, oximetry device 100 may include a dropout module 172. Oximetry device 100, using dropout module 172, may track dropouts in sensed pulse rate signal followed by an excessive difference in resumed pulse rate when compared to the last pulse rate value before the dropout. A dropout may be a missing pulse rate sample. Dropouts may be caused by multiple sources, for example, a sensor removal and re-application or excessive signal artifact(s) from patient motion at sensor site. When oximetry device 100 detects a dropout, oximetry device 100 may store a last valid pulse rate value and an associated timestamp in memory 120. Oximetry device 100, using dropout module 172, may determine when the pulse rate is sensed again within a predetermined time limit 504 and compare the new sensed pulse rate against the stored last valid pulse rate value prior to the dropout. If a difference between the current pulse rate value and the last valid pulse rate value is greater than a predetermined difference threshold 502(whether the current pulse rate value is higher or lower than baseline) - e.g., 30% of original baseline, oximetry device 100 may invalidate the incoming rate samples until the time limit is reached. If no resumed sensed sample is found until predetermined time limit threshold 504 is reached, oximetry device 100 may terminate the dropout module 172. Example predetermined time limit thresholds for dropout module 172 may include 30, 45 or 60 seconds, or any other values in between such values. In this manner, oximetry device 100, through the use of dropout module 172, may prevent large changes in HRV values due to step changes in pulse rate during a sensor disconnect or another dropout event.

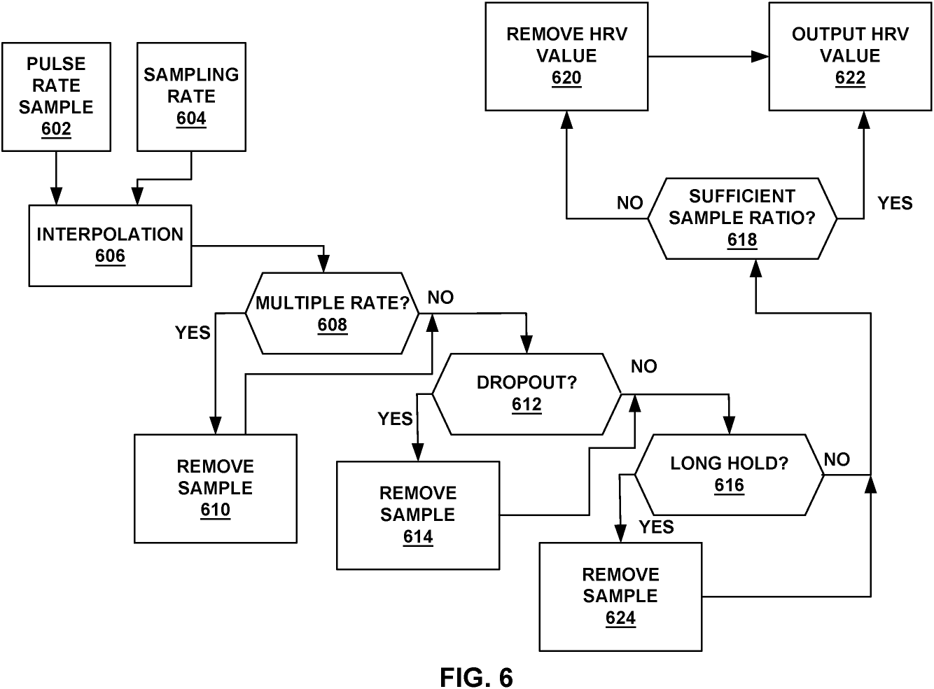

FIG. 6 is a flow diagram illustrating an example technique for determining and outputting an HRV value, in accordance with the techniques of this disclosure. Although FIG. 6 is described with respect to oximetry device 100 (FIG. 1), in other examples, another device or combination of devices may perform any part of the technique of FIG. 6.

Oximetry device 100 may receive a new pulse rate sample (602). For example, oximetry device 100 may receive a new pulse rate sample from another device or oximetry device 100 may sense a new pulse rate to receive the new pulse rate sample. Oximetry device 100 may retrieve from memory 132 a sampling rate at which the pulse rate is sampled (604). Oximetry device 100 may interpolate the input pulse rate to a fixed sample rate (606). Oximetry device 100 may determine whether the new pulse rate sample is a multiple rate of a baseline pulse rate (608). For example, oximetry device 100 may determine whether the new pulse rate sample is approximately twice or approximately one half of the baseline pulse rate.

If the new pulse rate sample is a multiple rate of the baseline pulse rate (the “YES” path from box 608), oximetry device 100 may remove the new pulse rate sample from the sensed pulse rate signal (610). If the new pulse rate sample is not a multiple rate of the baseline pulse rate (the “NO” path from box 608), oximetry device 100 may determine whether the new pulse rate sample is a dropout sample (612). For example, oximetry device 100 may detect that an expected pulse rate sample has not been received or sensed.

If the new pulse rate sample is a dropout sample (the “YES” path from box 612), oximetry device 100 may remove the new pulse rate sample from the sensed pulse rate signal (614). If the new pulse rate sample is not a dropout sample (the “NO” path from box 612), oximetry device 100 may determine whether the new pulse rate sample is a part of a long hold (616).

If the new pulse rate sample is a part of a long hold (the “YES” path from box 616), oximetry device 100 may remove the new pulse rate sample from the sensed pulse rate signal (624). If the new pulse rate sample is not a part of a long hold (the “NO” path from box 616), oximetry device 100 may determine whether there is a sufficient valid sample ratio to determine a meaningful HRV value (618). If there is a sufficient valid sample ratio to determine a meaningful HRV value (the “YES” path from box 618), oximetry device 100 may output the HRV value (622). If there is not a sufficient valid sample ratio to determine a meaningful HRV value (the “NO” path from box 618), oximetry device 100 may remove a determined HRV value (620) associated with the time period during which the insufficient samples were sensed and then output an HRV value (622) which does not include the removed HRV value.

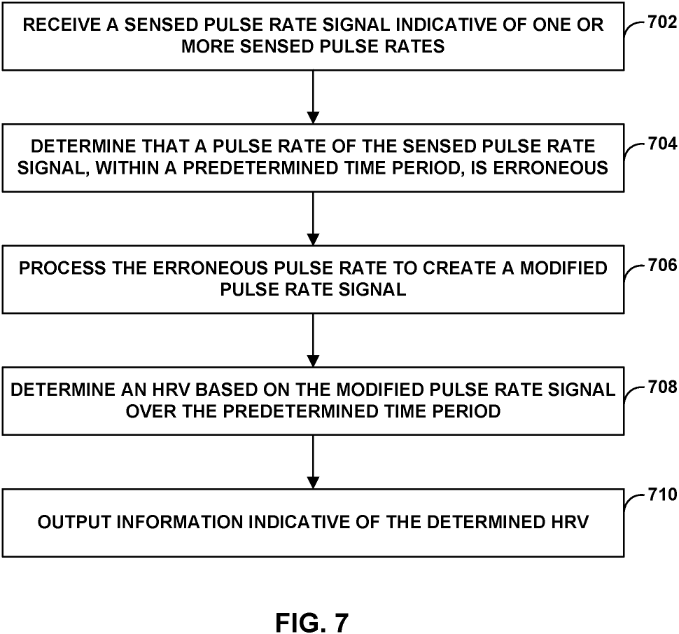

FIG. 7 is a flow diagram illustrating another example technique for determining and outputting an HRV value, in accordance with the techniques of this disclosure. While described as being performed by oximetry device 100 (FIG. 1), any part of the technique of FIG. 7 may be performed by another device or combination of devices.

Oximetry device 100 may receive a sensed pulse rate signal indicative of one or more sensed pulse rates (702). For example, detector 162 may sense light through tissue of a patient and sensing circuitry 140 may determine the sensed pulse rate signal.

Oximetry device 100 may determine that a pulse rate of the sensed pulse rate signal, within a predetermined time period, is erroneous (704). For example, oximetry device 100 may determine that a pulse rate of the sensed pulse rate signal is a multiple of a baseline rate, a dropout followed by an excessive difference in resumed pulse rate when compared to the last pulse rate value before the dropout, or a long hold pulse rate, within a time for determining an HRV value.

Oximetry device 100 may process the erroneous pulse rate to create a modified pulse rate signal (706). For example, oximetry device 100 may remove the erroneous pulse rate, may multiply the erroneous pulse rate by a multiplier (e.g., one half or two), or replace the erroneous pulse rate with a baseline pulse rate to create the modified pulse rate signal.

Oximetry device 100 may determine an HRV based on the modified pulse rate signal over the predetermined time period (708). For example, oximetry device 100 may calculate a standard deviation of the modified pulse rate signal over, for example, 5 minutes.

Oximetry device 100 may output information indicative of the determined HRV (710). For example, oximetry device 100 may display the HRV value on display 132.

In some examples, oximetry device 100 may process the erroneous pulse rate by applying a multiplier to the erroneous pulse rate or remove the erroneous pulse rate. In some examples, oximetry device 100 may determine that a ratio based on valid pulse rate samples within the predetermined time period is greater than or equal to a predetermined minimum ratio. In some examples, oximetry device 100 may determine the HRV based on the modified pulse rate signal over the predetermined time period and based on the ratio being greater than or equal to the predetermined minimum ratio.

In some examples, the one or more sensed pulse rates are sampled at a fixed sampling rate. In some examples, oximetry device 100 determines a baseline pulse rate. In some examples, oximetry device 100 determines that a pulse rate is erroneous comprises determining that the pulse rate is within a predetermined threshold of a multiple value of the baseline pulse rate.

In some examples, the HRV value is a first HRV value. In some examples, oximetry device 100, based on determining that the pulse rate is erroneous, stops providing a representation of a second HRV value. In some examples, oximetry device 100 determines that, at least one of, a current sensed pulse rate is within a predetermined range of the baseline pulse rate, or a predetermined timeout period beginning from the erroneous pulse rate has elapsed. In some examples, oximetry device 100, based on at least one of the current sensed pulse rate being within the predetermined range of the baseline pulse rate, or that the predetermined timeout period beginning from the erroneous pulse rate has elapsed, determines a third HRV value based on the modified pulse rate signal.

In some examples, oximetry device 100, based on determining that the pulse rate is within the predetermined multiple rate threshold of a multiple value of the baseline pulse rate, after a minimum elapsed time, determines a slope from a last baseline rate value to a first pulse rate value within a predetermined multiple rate threshold; determines that the slope is greater than or equal to a predetermined slope threshold; and based on the slope being greater than or equal to the predetermined slope threshold, stops outputting HRV values and deletes the slope from the sensed pulse rate signal.

In some examples, oximetry device 100 determines that the pulse rate is within a multiple rate threshold, and based on the pulse rate being within the multiple rate threshold, modifies the pulse rate by applying a multiplier to the pulse rate. In some examples, oximetry device 100 may determine that the pulse rate is not within a multiple rate threshold, and based on the pulse rate not being within the multiple rate threshold, delete the pulse rate.

In some examples, oximetry device 100 may monitor a ratio of pulse rate samples within the multiple rate threshold and based on the ratio of the pulse rate samples within the multiple rate threshold falling below a predetermined ratio threshold, stop the HRV determination and reset the HRV determination. In some examples, resetting the HRV determination includes restarting the predetermined time period.

In some examples, oximetry device 100 may store a last valid pulse rate value and an associated time stamp. Oximetry device 100 may determine that a difference between a value of a pulse rate received within a predetermined time limit and the last valid pulse rate is greater than a predetermined difference threshold. Oximetry device 100 may, based on the difference being greater than the predetermined difference threshold, invalidate incoming pulse rate samples until the predetermined time limit is reached.

In some examples, oximetry device 100 may determine that incoming pulse rate values have been constant or within a numerical tolerance of constant for a predetermined constant time limit threshold. Oximetry device 100 may, based on the determination that the incoming pulse rate values have been constant for the predetermined constant time limit threshold, discard incoming pulse rate values. Oximetry device 100 may determine that an incoming pulse rate value is different than the constant pulse rate values. Oximetry device 100 may resuming accepting incoming pulse rate values.

This disclosure includes the following non-limiting examples.

Example 1. A device for determining heart rate variability (HRV), the device comprising: a memory configured to store a sensed pulse rate signal indicative of one or more sensed pulse rates; and processor circuitry configured to: receive the sensed pulse rate signal; determine that a pulse rate of the sensed pulse rate signal, within a predetermined time period, is erroneous; process the erroneous pulse rate to create a modified sensed pulse rate signal; determine an HRV value based on the modified pulse rate signal over the predetermined time period; and output information indicative of the determined HRV value.

Example 2. The device of example 1, wherein to process the erroneous pulse rate, the processor circuitry is configured to apply a multiplier to the erroneous pulse rate or remove the erroneous pulse rate.

Example 3. The device of example 1 or example 2, wherein the processor circuitry is further configured to: determine that a ratio based on valid pulse rate samples within the predetermined time period is greater than or equal to a predetermined minimum ratio, and wherein to determine the HRV value, the processor circuitry is configured to determine the HRV value based on the modified pulse rate signal over the predetermined time period and based on the ratio being greater than or equal to the predetermined minimum ratio.

Example 4. The device of any of examples 1-3, wherein the one or more sensed pulse rates are sampled at a fixed sampling rate.

Example 5. The device of any of examples 1-4, wherein the processor circuitry is further configured to determine a baseline pulse rate.

Example 6. The device of example 5, wherein to determine that the pulse rate is erroneous, the processor circuitry is configured to determine that the pulse rate is within a predetermined multiple rate threshold of a multiple value of the baseline pulse rate.

Example 7. The device of example 5 or example 6, wherein the HRV value is a first HRV value, and wherein the processor circuitry is further configured to: based on determining that the pulse rate is erroneous, stop providing a representation of a second HRV value; determine that, at least one of, a current sensed pulse rate is within a predetermined range of the baseline pulse rate, or a predetermined timeout period beginning from the erroneous pulse rate has elapsed; based on at least one of the current sensed pulse rate being within the predetermined range of the baseline pulse rate, or that the predetermined timeout period beginning from the erroneous pulse rate has elapsed, determine a third HRV value based on the modified pulse rate signal.

Example 8. The device of example 6, wherein the processing circuitry is further configured to, based on determining that the pulse rate is within the predetermined multiple rate threshold: determine a slope from a last baseline rate value to a first pulse rate value within the predetermined multiple rate threshold, after a minimum elapsed time; determine that the slope is greater than or equal to a predetermined slope threshold; and based on the slope being greater than or equal to the predetermined slope threshold, stop outputting HRV values and delete the slope from the sensed pulse rate signal.

Example 9. The device of any of examples 6-8, wherein the processor circuitry is further configured to: determine that the pulse rate is within the predetermined multiple rate threshold; and based on the pulse rate being within the predetermined multiple rate threshold, modify the pulse rate by applying a multiplier to the pulse rate.

Example 10. The device of any of examples 6-8, wherein the processor circuitry is further configured to: determine that the pulse rate is not within the predetermined multiple rate threshold; and based on the pulse rate not being within the predetermined multiple rate threshold, delete the pulse rate.

Example 11. The device of example 9 or example 10, wherein the processor circuitry is further configured to: monitor a ratio of pulse rate samples within the predetermined multiple rate threshold; and based on the ratio of the pulse rate samples within the predetermined multiple rate threshold falling below a predetermined ratio threshold, stop the HRV determination and reset the HRV determination.

Example 12. The device of example 11, wherein to reset the HRV determination, the processing circuitry is configured to restart the predetermined time period.

Example 13. The device of any of examples 1-12, wherein the processing circuitry is further configured to: store a last valid pulse rate value and an associated time stamp; determine that a difference between a value of a pulse rate received within a predetermined time limit and the last valid pulse rate is greater than a predetermined difference threshold; and based on the difference being greater than the predetermined difference threshold, invalidate incoming pulse rate samples until the predetermined time limit is reached.

Example 14. The device of any of examples 1-13, wherein the processor circuitry is further configured to: determine that incoming pulse rate values have been constant or within a numerical tolerance of constant for a predetermined constant time limit threshold; based on the determination that the incoming pulse rate values have been constant for the predetermined constant time limit threshold, discard incoming pulse rate values; determine that an incoming pulse rate value is different than the constant pulse rate values; and resume accepting incoming pulse rate values.

Example 15. A method for determining heart rate variability (HRV), the method comprising: receiving a sensed pulse rate signal indicative of one or more sensed pulse rates; determining that a pulse rate of the sensed pulse rate signal, within a predetermined time period, is erroneous; processing the erroneous pulse rate to create a modified sensed pulse rate signal; determining an HRV value based on the modified pulse rate signal over the predetermined time period; and outputting information indicative of the determined HRV value.

Example 16. The method of example 15, wherein processing the erroneous pulse rate comprises applying a multiplier to the erroneous pulse rate or remove the erroneous pulse rate.

Example 17. The method of example 15 or example 16, further comprising: determining that a ratio based on valid pulse rate samples within the predetermined time period is greater than or equal to a predetermined minimum ratio, and wherein determining the HRV value comprises determining the HRV value based on the modified pulse rate signal over the predetermined time period and based on the ratio being greater than or equal to the predetermined minimum ratio.

Example 18. The method of any of examples 15-17, wherein the one or more sensed pulse rates are sampled at a fixed sampling rate.

Example 19. The method of any of examples 15-17, further comprising determining a baseline pulse rate.

Example 20. The method of example 19, wherein determining that the pulse rate is erroneous comprises determining that the pulse rate is within a predetermined multiple rate threshold of a multiple value of the baseline pulse rate.

Example 21. The device of example 19 or example 20, wherein the HRV value is a first HRV value, and wherein the method further comprises: based on determining that the received sensed pulse rate is erroneous, stopping providing a representation of a second HRV value; determining that, at least one of, a current sensed pulse rate is within a predetermined range of the baseline pulse rate, or a predetermined timeout period beginning from the erroneous pulse rate has elapsed; based on at least one of the current sensed pulse rate being within the predetermined range of the baseline pulse rate, or that the predetermined timeout period beginning from the erroneous pulse rate has elapsed, determining a third HRV value based on the modified pulse rate signal.

Example 22. The method of example 20, further comprising: based on determining that the pulse rate is within the predetermined multiple rate threshold of the multiple value of the baseline pulse rate: determining a slope from a last baseline rate value to a first pulse rate value within the predetermined multiple rate threshold, after a minimum elapsed time; determining that the slope is greater than or equal to a predetermined slope threshold; and based on the slope being greater than or equal to the predetermined slope threshold, stopping outputting HRV values and delete the slope from the sensed pulse rate signal.

Example 23. The method of any of examples 20-22, further comprising: determining that the pulse rate is within the predetermined multiple rate threshold; and based on the pulse rate being within the predetermined multiple rate threshold, modifying the pulse rate by applying a multiplier to the pulse rate.

Example 24. The method of any of examples 20-22, further comprising: determining that the pulse rate is not within the predetermined multiple rate threshold; and based on the pulse rate not being within the predetermined multiple rate threshold, deleting the pulse rate.

Example 25. The method of example 23 or example 24, further comprising: monitoring a ratio of pulse rate samples within the predetermined multiple rate threshold; and based on the ratio of the pulse rate samples within the predetermined multiple rate threshold falling below a predetermined ratio threshold, stopping the HRV determination and resetting the HRV determination.

Example 26. The method of example 25, wherein resetting the HRV determination comprises restarting the predetermined time period.

Example 27. The method of any of examples 15-26, further comprising: storing a last valid pulse rate value and an associated time stamp; determining that a difference between a value of a pulse rate received within a predetermined time limit and the last valid pulse rate is greater than a predetermined difference threshold; and based on the difference being greater than the predetermined difference threshold, invalidating incoming pulse rate samples until the predetermined time limit is reached.

Example 28. The method of any of examples 15-27, further comprising: determining that incoming pulse rate values have been constant or within a numerical tolerance of constant for a predetermined constant time limit threshold; based on the determination that the incoming pulse rate values have been constant for the predetermined constant time limit threshold, discarding incoming pulse rate values; determining that an incoming pulse rate value is different than the constant pulse rate values; and resuming accepting incoming pulse rate values.

Example 29. A non-transitory computer-readable storage medium storing instructions, which, when executed, cause processor circuitry to: receive a sensed pulse rate signal indicative of one or more sensed pulse rates; determine that a pulse rate of the sensed pulse rate signal, within a predetermined time period, is erroneous; process the erroneous pulse rate to create a modified sensed pulse rate signal; determine a heart rate variability (HRV) value based on the modified pulse rate signal over the predetermined time period; and output information indicative of the determined HRV value.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to devices 100 and 200, processing circuitry 110, 210, 214, and 216, memories 120 and 220, displays 132 and 232, sensing circuitries 140-142, circuitries 240 and 245, sensing devices 150, 151, 152, and 250, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in patient monitors, such as multiparameter patient monitors (MPMs) or other devices, remote servers, or other devices. The term “processor” or “processing circuitry” may generally refer to any of the foregoing logic circuitry, alone or in combination with other logic circuitry, or any other equivalent circuitry.

As used herein, the term “circuitry” refers to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. For example, any of the techniques or processes described herein may be performed within one device or at least partially distributed amongst two or more devices, such as between devices 100 and 200, processing circuitry 110, 210, 214, and 216, memories 120 and 220, sensing circuitries 140-142, and/or circuitries 240 and 245. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache). Elements of devices and circuitry described herein, including, but not limited to, devices 100 and 200, processing circuitry 110, 210, 214, and 216, memories 120 and 220, displays 132 and 232, sensing circuitries 140-142, circuitries 240 and 245, sensing devices 150-152 and 250 may be programmed with various forms of software. The one or more processors may be implemented at least in part as, or include, one or more executable applications, application modules, libraries, classes, methods, objects, routines, subroutines, firmware, and/or embedded code, for example.

Various examples of the disclosure have been described. Any combination of the described systems, operations, or functions is contemplated. These and other examples are within the scope of the following claims.

What is claimed is:

1. A device for determining heart rate variability (HRV), the device comprising:
a memory configured to store a sensed pulse rate signal indicative of one or more sensed pulse rates; and
processor circuitry configured to:
receive the sensed pulse rate signal;
determine that a pulse rate of the sensed pulse rate signal, within a predetermined time period, is erroneous;
process the erroneous pulse rate to create a modified sensed pulse rate signal;
determine an HRV value based on the modified pulse rate signal over the predetermined time period; and
output information indicative of the determined HRV value.

2. The device of claim 1, wherein to process the erroneous pulse rate, the processor circuitry is configured to apply a multiplier to the erroneous pulse rate or remove the erroneous pulse rate.

3. The device of claim 1, wherein the processor circuitry is further configured to:
determine that a ratio based on valid pulse rate samples within the predetermined time period is greater than or equal to a predetermined minimum ratio, and wherein to determine the HRV value, the processor circuitry is configured to determine the HRV value based on the modified pulse rate signal over the predetermined time period and based on the ratio being greater than or equal to the predetermined minimum ratio.

4. The device of claim 1, wherein the one or more sensed pulse rates are sampled at a fixed sampling rate.

5. The device of claim 1, wherein the processor circuitry is further configured to determine a baseline pulse rate.

6. The device of claim 5, wherein the HRV value is a first HRV value, and wherein the processor circuitry is further configured to:
based on determining that the pulse rate is erroneous, stop providing a representation of a second HRV value;
determine that, at least one of, a current sensed pulse rate is within a predetermined range of the baseline pulse rate, or a predetermined timeout period beginning from the erroneous pulse rate has elapsed;
based on at least one of the current sensed pulse rate being within the predetermined range of the baseline pulse rate, or that the predetermined timeout period beginning from the erroneous pulse rate has elapsed, determine a third HRV value based on the modified pulse rate signal.

7. The device of claim 5, wherein to determine that the pulse rate is erroneous, the processor circuitry is configured to determine that the pulse rate is within a predetermined multiple rate threshold of a multiple value of the baseline pulse rate.

8. The device of claim 7, wherein the processing circuitry is further configured to, based on determining that the pulse rate is within the predetermined multiple rate threshold, after a minimum elapsed time:
determine a slope from a last baseline rate value to a first pulse rate value within the predetermined multiple rate threshold;
determine that the slope is greater than or equal to a predetermined slope threshold; and
based on the slope being greater than or equal to the predetermined slope threshold, stop outputting HRV values and delete the slope from the sensed pulse rate signal.

9. The device of claim 7, wherein the processor circuitry is further configured to:
determine that the pulse rate is not within the predetermined multiple rate threshold; and
based on the pulse rate not being within the predetermined multiple rate threshold, delete the pulse rate.

10. The device of claim 7, wherein the processor circuitry is further configured to:
determine that the pulse rate is within the predetermined multiple rate threshold; and
based on the pulse rate being within the predetermined multiple rate threshold, modify the pulse rate by applying a multiplier to the pulse rate.

11. The device of claim 10, wherein the processor circuitry is further configured to:
monitor a ratio of pulse rate samples within the predetermined multiple rate threshold; and
based on the ratio of the pulse rate samples within the predetermined multiple rate threshold falling below a predetermined ratio threshold, stop the HRV determination and reset the HRV determination.

12. The device of claim 11, wherein to reset the HRV determination, the processing circuitry is configured to restart the predetermined time period.

13. The device of claim 1, wherein the processing circuitry is further configured to:
store a last valid pulse rate value and an associated time stamp;
determine that a difference between a value of a pulse rate received within a predetermined time limit and the last valid pulse rate is greater than a predetermined difference threshold; and
based on the difference being greater than the predetermined difference threshold, invalidate incoming pulse rate samples until the predetermined time limit is reached.

14. The device claim 1, wherein the processor circuitry is further configured to:
determine that incoming pulse rate values have been constant or less than a numerical tolerance for a predetermined constant time limit threshold;
based on the determination that the incoming pulse rate values have been constant or within a numerical tolerance of constant for the predetermined constant time limit threshold, discard incoming pulse rate values;
determine that an incoming pulse rate value is different than the constant pulse rate values; and
resume accepting incoming pulse rate values.

15. A method for determining heart rate variability (HRV), the method comprising:
receiving a sensed pulse rate signal indicative of one or more sensed pulse rates;
determining that a pulse rate of the sensed pulse rate signal, within a predetermined time period, is erroneous;
processing the erroneous pulse rate to create a modified sensed pulse rate signal;
determining an HRV value based on the modified pulse rate signal over the predetermined time period; and
outputting information indicative of the determined HRV value.

16. The method of claim 15, wherein processing the erroneous pulse rate comprises applying a multiplier to the erroneous pulse rate or remove the erroneous pulse rate.

17. The method of claim 15, further comprising:
determining that a ratio based on valid pulse rate samples within the predetermined time period is greater than or equal to a predetermined minimum ratio, and wherein determining the HRV value comprises determining the HRV value based on the modified pulse rate signal over the predetermined time period and based on the ratio being greater than or equal to the predetermined minimum ratio.

18. The method of claim 15, wherein the one or more sensed pulse rates are sampled at a fixed sampling rate.

19. The method of claim 15, further comprising determining a baseline pulse rate.

20. A non-transitory computer-readable storage medium storing instructions, which, when executed, cause processor circuitry to:
receive a sensed pulse rate signal indicative of one or more sensed pulse rates;
determine that a pulse rate of the sensed pulse rate signal, within a predetermined time period, is erroneous;
process the erroneous pulse rate to create a modified sensed pulse rate signal;
determine a heart rate variability (HRV) value based on the modified pulse rate signal over the predetermined time period; and
output information indicative of the determined HRV value.
